(19) United States
(12) Patent Application Publication
(10) Pub. No.: US 2026/0354927 A1
(43) Pub. Date: Nov. 12, 2026

(54) DEVICES AND METHODS FOR DETECTING INTERNAL ABDOMINAL HERNIA

(71) Applicant: Mayo Foundation for Medical Education and Research, Rochester, MN (US)

(72) Inventors: Omar M. Ghanem, Rochester, MN (US); Brody R. Stewart, Rochester, MN (US)

(21) Appl. No.: 19/670,480

(22) Filed: May 7, 2026

Related U.S. Application Data
(60) Provisional application No. 63/801,466, filed on May 7, 2025.

(51) Int. Cl. A61B 90/00; A61B 17/04; A61B 17/00; A61B 17/06

(52) U.S. Cl. CPC A61B 90/39; A61B 17/0487; A61B 2017/00477; A61B 2017/06176; A61B 2090/3908; A61B 2090/3966; A61B 2090/3991; A61B 2503/40

(57) ABSTRACT

This document provides devices and methods for detecting internal abdominal hernia in a subject (e.g., human or animal). For example, a device disclosed herein can include a first strand comprising a plurality of integrated radiopaque markers having a first geometric shape, a second strand comprising a plurality of integrated radiopaque markers having a second geometric shape, wherein the second geometric shape is different from the first geometric shape, and a coupling member configured to join the first strand and the second strand such that the first strand and the second strand are positioned on opposite sides of a defect within an abdominal cavity of a subject, wherein the first geometric shape and the second geometric shape are configured to allow visual differentiation between the first strand and the second strand under medical imaging.

### CROSS REFERENCE TO RELATED APPLICATIONS

The application claims the benefit of priority to U.S. Patent Application No. 63/801,466, filed on May 7, 2025, the entire contents of which are hereby incorporated by reference.

### TECHNICAL FIELD

This document relates to devices and methods for detecting internal abdominal hernia. For example, this document relates to devices and methods for detecting internal hernias after intra-abdominal surgical procedures such as bowel resection operations.

### Background Information

Internal hernias are a complication that can occur after intra-abdominal surgeries that involve rearrangement of the gastrointestinal tract. Intra-abdominal surgeries can include bariatric surgeries, colorectal surgeries, gastrointestinal surgeries, oncologic resections, abdominal trauma surgical procedures, hernia repairs, etc. During surgery, spaces or defects can be unintentionally created within the abdominal cavity. If a loop of bowel herniates through one of these spaces, it can become trapped. The trapped bowel can lead to an obstruction and or a reduction in blood flow that causes ischemia and necrosis. Because internal hernias may not present with clear external signs, diagnosis is often challenging.

In some cases, diagnosis of herniated bowel is achieved using imaging such as computed tomography (CT) scans, X-ray, Upper Gastrointestinal (UGI) series, and magnetic resonance imaging (MRI). However, true herniation can be difficult to distinguish from other, less alarming, conditions. Due to the risk of rapid progression to bowel ischemia and necrosis, clinical suspicion often leads to a low threshold for surgical exploration. Treatment involves urgent surgical intervention to reduce the hernia and assess the viability of the bowel.

### SUMMARY

This document describes devices and methods for detecting internal abdominal hernia in a subject (e.g., human or animal) after intra-abdominal surgery such as bowel resection, transection, or shuffling operations. Non limiting examples of intra-abdominal surgeries include bariatric surgeries (e.g., gastric bypass, biliopancreatic diversion with duodenal switch, omega loop bypass or duodenal switch operations), colorectal surgeries (e.g., low anterior resection, right hemicolectomy, sigmoid colectomy), gastrointestinal surgeries (e.g., small bowel resection, gastrectomy, duodenal switch), oncologic resections (e.g., colectomy for colon cancer, gastrectomy for gastric cancer, debulking surgery for peritoneal carcinomatosis), abdominal trauma surgical procedures (e.g., exploratory laparotomy with bowel repair, damage control surgery with temporary closures), and hernia repairs (e.g., parastomal hernia repair, incisional hernia repair, ventral hernia repair).

Internal hernias following intra-abdominal surgery are a significant complication that poses a significant burden on patients and the healthcare system. In the United States, more than 300,000 surgeries are performed annually for bowel obstructions, with internal hernias accounting for an estimated 5.8% of cases. Diagnostic uncertainty often leads to unnecessary surgical explorations, as mesenteric spaces that are created during surgery may not be visible on imaging. With over 15,000 internal hernia cases each year and rates of procedures like Roux-en-Y gastric bypass increasing, there is a need to close this diagnostic gap to prevent delays in care, reduce morbidity, and avoid unnecessary operations.

Intra-abdominal surgeries often involve rearranging or mobilizing sections of the bowel. These surgical steps create potential spaces within the abdominal cavity where loops of intestine can later become trapped. Bariatric surgeries such as gastric bypass, biliopancreatic diversion with duodenal switch, omega loop bypass or duodenal switch operations are particularly associated with the formation of mesenteric defects that predispose patients to internal hernias. Similarly, mobilization during colorectal and gastrointestinal surgeries can leave openings that, over time, can allow for herniation. While these surgical modifications are important for treating underlying disease, they introduce ongoing risks that can be difficult to detect once the immediate recovery period has passed.

Approaches to managing mesenteric defects vary among surgeons. Some choose not to close the defects, while others opt to use absorbable or permanent sutures. However, even when the defects are closed intraoperatively with absorbable or permanent sutures, internal hernias still occur. For example, even when the defects are closed intraoperatively using permanent, non-absorbable sutures, hernias still occur in an estimated 3–5% of patients (Gormsen et al. Br J Surg. 2021 Mar 12;108(2)).

Patients who develop internal hernias often present with vague and nonspecific symptoms, such as intermittent abdominal pain, nausea, vomiting, or signs of bowel obstruction. Although imaging studies like CT scans and MRI are used to evaluate these symptoms, they often fail to clearly differentiate between an internal hernia and more benign causes, such as adhesions or transient digestive issues. Because missing an internal hernia can have devastating consequences—including bowel ischemia, necrosis, and death—physicians often choose to intervene surgically based on suspicion rather than definitive evidence. This diagnostic uncertainty results in a high number of negative explorations, exposing patients to unnecessary surgery and its associated risks. Bariatric patients are particularly vulnerable due to changes in intra-abdominal anatomy and fat distribution following significant weight loss, further complicating the diagnostic landscape.

Some embodiments of the devices and methods described herein can provide one or more of the following advantages and technical effects. First, some embodiments described herein disclose a marker suture with strands integrated with radiopaque markers that are placed on either side of a defect. The marker suture disclosed herein is a diagnostic tool for detecting an internal hernia associated with a peritoneal or mesenteric defect. For example, a defect can include a potential space created by the intra-abdominal surgery, such as a mesenteric defect or peritoneal defect, or other surgically created opening. In some examples, the peritoneal or mesenteric defects are closed with an anchor suture (e.g., an absorbable or nonabsorbable suture). In some instances, a surgeon can choose not to close a defect. The marker sutures disclosed herein are designed to be highly visible on imaging by providing a clear outline radiographically, regardless of the choice of the surgeon to close or not close the defect. For example, the marker sutures disclosed herein can be positioned around a defect, whether they are closed with absorbable suture, closed with nonabsorbable suture, or not closed. In this way, if bowel passes between the strands integrated with the radiopaque beads, it indicates the presence of an internal hernia. If no bowel is seen between the markers, an internal hernia can be effectively ruled out, which allows for more conservative, non-surgical management. If a bowel is detected, intervention can occur quickly.

Second, some embodiments described herein disclose a marker suture with integrated radiopaque facets formed as part of a non-dissolving surgical thread. The marker sutures are constructed by integrating radiopaque facets on non-dissolving surgical thread, allowing them to remain in place for the duration of the patient's life. As a result, changes in the abdominal cavity over time, such as those associated with significant postoperative weight loss or tissue remodeling—can still be monitored using the persistent and identifiable marker sutures without the risk of degradation or loss of signal.

Third, some embodiments described herein disclose a marker suture having first and second strands, each incorporating radiopaque facets of different shapes. The use of distinct geometric shapes between the two strands provides a visual distinction during imaging. This enables a clinician to determine whether the marker sutures have become dislodged, twisted, or otherwise displaced relative to their original positioning. This shape differentiation enhances diagnostic confidence and reduces the risk of misinterpretation when evaluating postoperative imaging.

Fourth, some embodiments described herein disclose that the radiopaque facets integrated into the marker sutures can be formed from any suitable metallic or non-metallic radiopaque material. In some examples, the facets can be made from titanium or other biocompatible materials that do not interfere with MRI. This construction allows patients to safely undergo MRI procedures while still retaining the ability to visualize the marker sutures using other imaging modalities such as X-ray or CT scans.

Fifth, some embodiments described herein disclose marker sutures that are not subjected to meaningful tension across the defect or when surgically positioned. Unlike earlier technologies where radiopaque elements were integrated into absorbable sutures intended to approximate and close tissue under tension, the marker sutures described herein are positioned passively to serve as durable, long-term markers. Because they are not under tension and/or do not facilitate mechanical closure or positioning of the defect, the marker sutures are less prone to migration, material fatigue, or deformation. This approach provides a distinct functional advantage, allowing the markers to remain stable and easily identifiable on imaging without being compromised by dynamic forces that can act on load-bearing closure sutures.

Sixth, some embodiments described herein disclose that the radiopaque facets integrated into the marker sutures are designed with multi-faceted geometric shapes. The use of multi-faceted structures increases the surface area of contact between the marker and the covering surrounding tissue. Distributing any mechanical forces over a broader area of the multi-faceted design helps avoid the problem of localized pressure points that could otherwise lead to tissue erosion. This configuration reduces the risk of the marker sutures cutting through delicate tissues, thereby enhancing their long-term stability and reliability as imaging markers.

One aspect of this document features devices and methods for marker sutures and positioning of a marker suture assembly during intra-abdominal surgery. In some example embodiments, devices can include, or consist essentially of, a marker suture assembly comprising a first strand comprising a plurality of integrated radiopaque markers having a first geometric shape, a second strand comprising a plurality of integrated radiopaque markers having a second geometric shape, wherein the second geometric shape is different from the first geometric shape; and a coupling member configured to join the first strand and the second strand such that the first strand and the second strand are positioned on opposite sides of a defect within an abdominal cavity of a subject, wherein the first geometric shape and the second geometric shape are configured to allow visual differentiation between the first strand and the second strand under medical imaging.

In another implementation, devices can include, or consist essentially of, a marker suture assembly comprising a first strand comprising a plurality of integrated radiopaque markers having a first geometric shape configured for positioning on a first side of a defect within an abdominal cavity of a subject, a second strand comprising a plurality of integrated radiopaque markers having a second geometric shape configured for positioning on a second side of the defect within the abdominal cavity of the subject, wherein the second geometric shape is different from the first geometric shape, and a coupling member configured to join the first strand and the second strand, wherein the coupling member is further configured to maintain the first strand and the second strand in a joined configuration while permitting rotational movement of the first strand relative to the second strand after coupling.

In some implementations, devices disclosed herein can include one or more of the following features. In some embodiments, the first geometric shape is a multi-faceted shape. In some embodiments, the second geometric shape is a cylindrical shape. In some embodiments, the integrated radiopaque markers of the first strand and the integrated radiopaque markers of the second strand are made from one or more materials selected from the group consisting of barium sulfate, tantalum oxide, zirconium dioxide, bismuth subcarbonate, bismuth trioxide, iodinated polymers, hydroxyapatite, calcium-based compounds, and titanium dioxide. In some embodiments, the integrated radiopaque markers of the first strand and the integrated radiopaque markers of the second strand are made from titanium dioxide. In some embodiments, the defect is one or more of a mesenteric defect, a peritoneal defect, a surgically created opening following bowel resection, a surgical anastomosis site, or a fascial defect created during hernia repair. In some embodiments, the subject is a human or animal and the marker suture assembly is placed during intra-abdominal surgery.

In some embodiments, the coupling member comprises a flexible body configured to be bent or pressed closed to join the first strand and the second strand. In some embodiments, the coupling member is formed in a ring shape configured to encircle and secure portions of the first strand and the second strand. In some embodiments, the first strand and the second strand each further comprise a linking member configured to attach to the coupling member. In some embodiments, each of the linking members are configured to allow rotational movement of the first strand relative to the coupling member after attachment. In some embodiments, the coupling member and the respective linking members are collectively configured to maintain an attachment of the first strand and the second strand while permitting relative rotational movement between the first strand, the second strand, and the coupling member. In some embodiments, the coupling member comprises a post extending from a first side and a hole formed through a second side, the hole being sized to engage the post with an interference fit to join the first strand and the second strand.

In some example embodiments, methods can include, comprise, or consist essentially of, a method of positioning a marker suture assembly during intra-abdominal surgery, the method comprising passing a first strand of the marker suture assembly through mesenteric or peritoneal tissue on a first side of a defect within an abdominal cavity, passing a second strand of the marker suture assembly through mesenteric or peritoneal tissue on a second side of the defect, securing the first strand and the second strand together using a coupling member, and positioning the first strand and the second strand with a length and slack configured to accommodate natural movement of abdominal tissues, thereby maintaining a spatial relationship across the defect while supporting postoperative monitoring.

In some implementations, the methods disclosed herein can include, comprise, or consist essentially of one or more of the following. In some embodiments, the first strand and the second strand are positioned with sufficient slack to accommodate postoperative changes in the abdominal cavity, including peristalsis, respiration, weight loss, or tissue remodeling. In some embodiments, the method further comprises imaging the first strand and the second strand after placement, wherein the imaging detects differently shaped integrated radiopaque markers on the first strand and the second strand to assess the spatial positioning across the defect. In some embodiments, the intra-abdominal surgery is performed using one or more modalities selected from open surgery, laparoscopic surgery, endoscopic surgery, or robotic-assisted surgery. In some embodiments, the defect comprises an anchor suture formed from dissolvable surgical thread used to close the defect. In some embodiments, the coupling member is configured to be bent or pressed closed during surgery to join the first strand and the second strand while permitting relative movement between the first strand and the second strand after placement.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 depicts an illustration of an example marker suture with integrated radiopaque markers as disclosed herein positioned on either side of a defect in an abdominal cavity.

FIG. 2 depicts a perspective view of an example marker suture with integrated radiopaque markers.

FIG. 3 depicts a perspective view of an example radiopaque marker with a multi-faceted geometric shape.

FIG. 4 depicts a perspective view of another example radiopaque marker with an elongated geometric shape.

FIG. 5 depicts a perspective view of a coupling member linking a first strand and a second strand of the marker suture.

FIG. 6A depicts a perspective view of another example of a coupling member in a disconnected configuration.

FIG. 6B depicts a perspective view of the example coupling member of FIG. 6A in a connected configuration linking a first strand and a second strand.

FIG. 7 shows a flow chart of an example method for positioning a marker suture assembly during intra-abdominal surgery.

FIG. 8 depicts a schematic view of an example system for forming a braided multi-strand marker suture.

FIG. 9 depicts a perspective view of an example radiopaque marker configured as a coil.

FIG. 10 depicts a perspective view of another example radiopaque marker configured as a spring.

FIG. 11 depicts a perspective view of another example radiopaque marker configured as a spiral.

### DETAILED DESCRIPTION

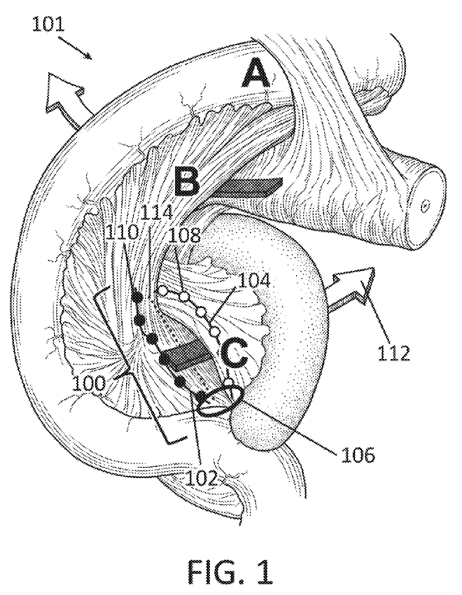

FIG. 1 depicts an illustration of an example marker suture 100 with integrated radiopaque markers 108 and 110 as disclosed herein, positioned on either side of a defect 114 in an abdominal cavity 101. In the illustrated example of FIG. 1, the marker suture 100 includes a first strand 102 and a second strand 104. The first strand 102 includes a set of integrated radiopaque markers 110, and the second strand 104 includes a separate set of integrated radiopaque markers 108. The integrated radiopaque markers 108 and 110 are affixed or embedded along the length of their respective strands in a predetermined pattern or spacing. These radiopaque markers can be formed from a variety of radiopaque materials and configured to have distinct geometries, such as cylindrical or multifaceted shapes, to improve visibility and orientation on medical imaging.

The first strand 102 and the second strand 104 can be operatively connected using a coupling member 106. The coupling member 106 can be a mechanical coupling. In some embodiments, the coupling member 106 can be configured to maintain a spatial relationship between the two strands without placing the marker suture 100 under tension. For example, the marker suture 100 is configured to be positioned such that the first strand 102 and the second strand 104 are located on opposing sides of a defect 114, with the defect 114 disposed between them. In such examples, the coupling member 106 connects the two strands 102 and 104 to maintain position but allow movement as the patient move their body. The marker suture 100 is not intended to serve as a primary closure device. For example, the marker suture 100 functions as a diagnostic aid to allow postoperative monitoring of the defect 114 through medical imaging.

In some embodiments, the defect 114 is closed with an anchor suture . For example, defects can result from surgical procedures including, but not limited to, gastric bypass, small bowel resection, or colorectal resection. An anchor suture can comprise dissolvable surgical thread (e.g., polyglycolic acid (PGA), polyglactin 910 (e.g., VICRYL™), polydioxanone (e.g., PDS II™), poliglecaprone (e.g., MONOCRYL™), and other commercially available absorbable synthetic threads used in soft tissue approximation) and can be used by the surgeon to approximate adjacent mesenteric or peritoneal tissue and eliminate a surgically created potential space. In some cases, a surgeon can close a defect 114 with non-dissolvable means (e.g., non-dissolvable thread or barbed sutures).

In contrast, the first strand 102 and the second strand 104 is made from surgical thread that is not dissolvable (e.g., any biocompatible non-dissolvable surgical thread). Non-limiting example of non-dissolvable surgical threads includes polypropylene, polyester, nylon, expanded polytetrafluoroethylene (ePTFE), braided, multi-filament, and single filament.

The non-dissolvable surgical thread facilitates that the marker suture 100 remains in place to allow postoperative monitoring of the defect 114. For example, in a case where the surgeon has closed the defect 114 with dissolvable thread, the marker suture 100 remains in place after the dissolvable thread of the anchor suture has dissipated. In another example, in a case where the surgeon has closed the defect 114 with an anchor suture made from non-dissolvable thread, the marker suture 100 will still remain in place around the defect 114 and can help diagnose a hernia in the event of a failure of the non-dissolvable thread used to close the defect 114.

The marker suture 100 can be positioned in the same operative field such that the first strand 102 and second strand 104 are woven through tissue, flanking the defect 114 (e.g., anchor suture) and remain passively in place. This configuration allows the marker suture 100 to persist in the body as a spatial reference for identifying potential internal herniation using imaging modalities such as CT or MRI. In operation, the example shown in FIG. 1 illustrates the surgical placement of the marker suture 100 following a gastric bypass procedure, such as a Roux-en-Y configuration. In the illustrated example, segment A represents a portion of the small intestine, while region B represents mesenteric tissue that has been divided or opened during the creation of the bowel anastomosis. Region C (shown adjacent to the defect 114 (e.g., anchor suture)) represents the potential space or defect that remains after the creation of the jejunojejunostomy. Arrow 112 indicates a potential example path of internal herniation, illustrating how a loop of bowel can migrate through the defect 114, which could result in bowel entrapment, obstruction, or strangulation.

In some implementations, each of the first strand 102 and the second strand 104 is passed through the mesenteric tissue flanking the defect 114 (e.g., anchor suture). The mesenteric tissue is the membranous structure that suspends the intestines and contains blood vessels, nerves, and lymphatics. The mesenteric tissue it is shown in FIG. 1 surrounding the anastomosis and extending between bowel segments. The strands 102 and 104 are positioned so that they span the defect 114, with each strand on opposite sides. After the strands 102 and 104 are passed through the mesentery, they are joined together using the coupling member 106. The coupling member 106 can secure the marker suture 100 without exerting tension across the tissue. In this way, the radiopaque markers 108 and 110 remain embedded within the mesentery to serve as imaging references in the event that internal herniation occurs postoperatively.

In some embodiments, the first strand 102 and the second strand 104 are positioned using a suture needle. For example, suitable needles can include curved SH (small half-circle) taper point needles, for example in sizes ranging from 2-0 to 4-0, depending on the thickness of the mesenteric tissue and the surgical approach (e.g., open versus laparoscopic). The selected needle size and thread diameter can be tailored to lessen trauma to the surrounding tissue while ensuring durable placement of the marker suture.

The surgical placement of marker suture 100 can be performed with any appropriate surgical modality. Placement can occur during the initial surgery or after surgery to monitor healing, assess the stability of a repaired defect, or evaluate for recurrent herniation. The marker suture 100 can be adapted for use in a variety of surgical approaches. For example, the marker suture 100 can be adapted for use during open surgical procedures, laparoscopic procedures, endoscopic procedures, and/or robotic-assisted procedures, allowing flexibility based on the clinical setting and the surgeon’s preferred technique.

In some implementations, during open surgical procedures, the surgeon can directly visualize the mesenteric or peritoneal defect following bowel manipulation or anastomosis. In an example operation, a first strand 102 and second strand 104 can be manually passed through opposing sides of the mesenteric tissue using suture needles resulting in placement on either side of the defect 114 (e.g., anchor suture). After threading through the tissue, the strands can be joined using the coupling member 106, leaving the integrated radiopaque markers 108 and 110 embedded in stable positions for future imaging evaluation.

In another example, in laparoscopic procedures, placement of the marker suture 100 can be accomplished through laparoscopic ports using less invasive instruments. In such implementations, a laparoscopic needle driver can be used to pass the first strand 102 and second strand 104 through the mesenteric tissue on opposite sides of the defect 114. After threading through the tissue, the strands 102 and 104 can be joined using the coupling member 106, leaving the integrated radiopaque markers 108 and 110 embedded in stable positions for future imaging evaluation. In this example, visualization can be maintained with the laparoscope to ensure accurate positioning of the markers across the defect.

In another example, in endoscopic procedures, a marker suture 100 can be deployed using a flexible endoscopic suturing system. In such cases where defects are accessible endoluminal, such as following certain gastric or intestinal interventions, an endoscopic suturing device can pass the first strand 102 and second strand 104 across the identified defect 114. After threading through the tissue, the strands can be joined using the coupling member 106, leaving the integrated radiopaque markers 108 and 110 embedded in stable positions for future imaging evaluation. For example, the coupling member 106 can be engaged through the working channel of the endoscope to complete marker suture 100 placement without the need for external incisions.

In another example, in robotic-assisted procedures, placement of marker suture 100 can be facilitated by robotic arms equipped with, for example, needle drivers. In some embodiments, the robotic arms can include three-dimensional visualization. In some embodiments, the surgeon can manipulate the robotic instruments to pass the first strand 102 and second strand 104 through tissue on opposing sides of the defect 114. The coupling member 106 can be joined using robotic graspers to secure the strands in place.

In some embodiments, the placement of the marker suture 100 is selectively performed to avoid introducing tension across the surrounding tissue. For example, when positioning the first strand 102 and the second strand 104, the tissue for positioning is selected to place the strands loosely through opposing sides of the tissue flanking the defect 114, rather than pulling the strands 102 and 104 taut. By decreasing tension, the marker suture 100 can adapt to natural movements of the abdominal cavity, such as shifts caused by respiration, digestion, or postoperative changes in body mass, without exerting stress on the underlying tissue.

For example, during placement, the first strand 102 and the second strand 104 can be woven through the tissue (e.g., mesenteric or peritoneal tissue) in a manner that surrounds the defect 114 therebetween but does not constrict the defect 114. The strands 102 and 104 can be loosely looped or passed through tissue (e.g., mesenteric or peritoneal tissue) planes with enough slack to accommodate physiological motion. In this way, the marker suture 100 effectively frames the defect 114 without applying a mechanical load that could otherwise compromise tissue integrity. Compromised tissue integrity can lead to migration of the first strand 102 and the second strand 104. Thus, avoiding the migration of the first strand 102 and/or the second strand 104 is important to visualize the defect 114. This approach provides an advantage because mesenteric and peritoneal tissues are delicate, highly vascularized, and prone to tearing when subjected to concentrated forces.

In contrast, if the marker suture 100 were placed under significant tension, several detrimental effects could occur. For example, radiopaque markers 108 and 110 are integrated along the strands 102 and 110. When pulled against fragile tissue (e.g., mesenteric or peritoneal tissue) under stress, the radiopaque markers 108 and 110 integrated along the strands 102 and 110 could migrate or cut through the tissue over time. Additionally, strands 102 and/or 104 placed under tension can themselves shift or tear through the tissue, leading to loss of positional accuracy and reduced diagnostic reliability. As such, tensioned placement would undermine the intended long-term function of the marker suture 100 by introducing instability. The devices and methods described herein provide for tension-free placement that can preserve both the integrity of the surrounding tissue and the spatial fidelity of the marker suture 100 for the lifetime of the patient.

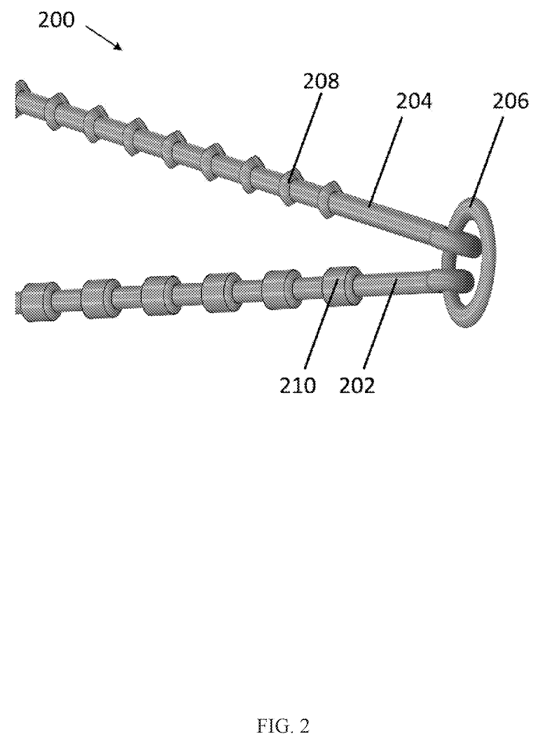

FIG. 2 depicts a perspective view of an example marker suture 200 with integrated radiopaque markers 208 and 210. The example marker suture 200 can be analogous to the example marker suture 100 described in connection with FIG. 1. The first strand 202 includes a set of integrated radiopaque markers 210, and the second strand 204 includes a separate set of integrated radiopaque markers 208. The first strand 202 and the second strand 204 can be connected by coupling member 206. The integrated radiopaque markers 208 and 210 are affixed or embedded along the length of their respective strands in a predetermined pattern or spacing.

The first strand 202 and the second strand 204 can be of any suitable length. In some embodiments, the length of the first strand 202 and/or second strand 204 is about 10 mm to about 50 mm. For example, the first strand 202 and/or the second strand 204 can be about 10 mm to about 50 mm, about 10 mm to about 40 mm, about 10 mm to about 30 mm, or about 10 mm to about 20 mm. For example, the first strand 202 and/or the second strand 204 can be about 20 mm to about 50 mm, about 30 mm to about 50 mm, or about 40 mm to about 50 mm. For example, the first strand 202 and/or the second strand 204 can be about 15 mm to about 35 mm, about 20 mm to about 35 mm, or about 25 mm to about 35 mm. For example, the first strand 102 and/or the second strand 104 can be about 25 mm.

In some embodiments, the integrated radiopaque markers 208 and the integrated radiopaque markers 210 can be spaced along the first strand 202 and the second strand 204 at regular intervals. For example, the interspacing between adjacent radiopaque markers can be about 2 mm to about 10 mm, about 3 mm to about 8 mm, or about 4 mm to about 6 mm, depending on the desired imaging resolution and the anatomical area being monitored.

The integrated radiopaque markers 208 and/or the integrated radiopaque markers 210 can be made from any radiopaque material. For example, the integrated radiopaque markers 208 and/or the integrated radiopaque markers 210 can be made from any suitable metal or non-metal radiopaque material. In some embodiments, the integrated radiopaque markers 208 and/or the integrated radiopaque markers 210 can be made from titanium dioxide. In some embodiments, integrated radiopaque markers 208 and/or the integrated radiopaque markers 210 can be made from barium sulfate, tantalum oxide, zirconium dioxide, bismuth subcarbonate, bismuth trioxide, iodinated polymers, hydroxyapatite, calcium-based compounds, or titanium dioxide.

In some embodiments, the integrated radiopaque markers 208 are of a different geometric shape than the integrated radiopaque markers 210. Such embodiments can provide the clinician with spatial information. For example, radiopaque markers 208 can be configured in a first geometric shape, and radiopaque markers 210 can be configured in a second geometric shape, such that each strand can be visually differentiated under imaging modalities such as CT or MRI. For example, when positioned during surgery, the first strand 202 with radiopaque markers 208 can be placed to the left side of the defect (e.g., defect 114), while the second strand 204 carrying radiopaque markers 210 can be placed to the right side of the defect (e.g., defect 114). During postoperative imaging, the clinician can observe the relative positioning of the different geometric shapes to assess the status of the defect (e.g., the surgical site). If imaging shows bowel interposed between the differently shaped markers, or if the markers have become displaced relative to each other, it can indicate that a herniation has occurred through the defect (e.g., defect 114). Conversely, if the geometric pattern and spatial relationship between the first strand 202 and second strand 204 remains intact, it suggests that no herniation or migration has occurred and that the repaired or monitored defect (e.g., defect 114) remains stable.

The example of FIG. 2 depicts the coupling member 206 as a ring shape though other shapes are possible. The coupling member 206 can be of any suitable size for example, having an outer diameter of about 2 mm to about 10 mm, about 3 mm to about 8 mm, about 4 mm to about 7 mm, or about 5 mm to about 6 mm. In some embodiments, the coupling member 206 can have a cross-sectional thickness of about 0.5 mm to about 2 mm, about 0.8 mm to about 1.5 mm, or about 1 mm. The size and thickness of the coupling member 206 can be selected to balance flexibility with secure retention of the first strand 202 and the second strand 204 without introducing significant tension across the defect.

The coupling member 206 can be made from any suitable metal or non-metal radiopaque material. In some embodiments, coupling member 206 can be made from barium sulfate, tantalum oxide, zirconium dioxide, bismuth subcarbonate, bismuth trioxide, iodinated polymers, hydroxyapatite, calcium-based compounds, or titanium dioxide.

In some embodiments, the coupling member 206 can include a break or gap that allows the ring to be opened and then bent or pressed closed during surgery. The bending of the closure can secure the first strand 202 and the second strand 204 without the need for additional fastening devices. This configuration can facilitate quick and reliable intraoperative placement while decreasing manipulation of the surrounding tissue.

FIG. 3 depicts a perspective view of an example radiopaque marker 308 with a multi-faceted geometric shape. The example radiopaque marker 308 shows a faceted structure with multiple flat surfaces 320 arranged at various angles, forming a generally polyhedral body. This example embodiment provides an advantage over spherical shapes, which would otherwise create a single point of contact with the peritoneum, concentrating pressure on a small area and increasing the risk of tissue erosion. By contrast, the multi-faceted design distributes contact forces across a greater surface area, thereby reducing localized pressure and decreasing the likelihood of erosion or migration through the delicate tissue over time.

In some embodiments, the radiopaque marker 308 can include a central lumen 309 extending axially through its body. This lumen 309 can accommodate the strands (e.g., first strand 102, 202, or second strand 104, 204). In some embodiments, the strands 102, 202, 104, 204 can integrate the example radiopaque marker 308. For example, the radiopaque marker 308 can be molded directly onto the strand during manufacturing, threaded onto the strand and secured in place using a friction fit or adhesive, embedded partially within the material of the strand itself during extrusion or fabrication, or attached using mechanical features such as crimps, barbs, or locking collars that engage the surface of the strand.

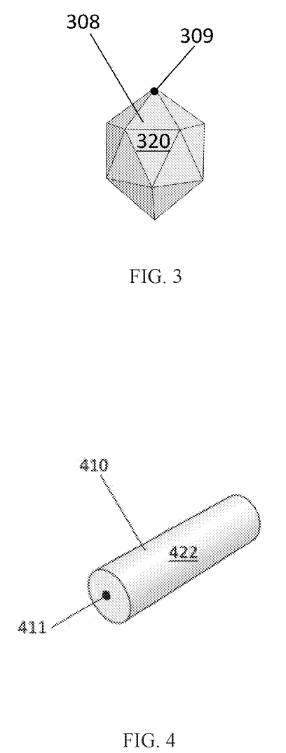

FIG. 4 depicts is a perspective view of another example radiopaque marker 410 with an elongated geometric shape. The example radiopaque marker 410 shows an elongated, generally cylindrical structure. The longitudinal surface 422 of the generally cylindrical body creates relatively large surface area that distributes contact forces. This provides an advantage over spherical which present a smaller surface area at points of tissue contact, which may concentrate pressure along a narrower region of the tissue and increase the risk of localized tissue erosion over time.

In some embodiments, the radiopaque marker 410 can include a central lumen 411 extending axially through its body. This lumen can accommodate the strands (e.g., first strand 102, 202, or second strand 104, 204). In some embodiments, the strands 102, 202, 104, 204 can integrate the example radiopaque marker 410. For example, the radiopaque marker 410 can be molded directly onto the strand during manufacturing, threaded onto the strand and secured in place using a friction fit or adhesive, embedded partially within the material of the strand itself during extrusion or fabrication, or attached using mechanical features such as crimps, barbs, or locking collars that engage the surface of the strand.

Referring to FIGS. 3 and 4 , in some embodiments, in some embodiments, different geometric shapes can be selectively positioned on the first strand (e.g., first strand 102 and 202) and the second strand (e.g., second strand 104 and 204) to visually distinguish them during imaging. For example, a series of multi-faceted radiopaque markers as shown in FIG. 3 can be integrated along the first strand, while a series of cylindrical radiopaque markers as shown in FIG. 4 can be integrated along the second strand. In this manner, the geometric difference between the markers provides a clear visual cue to differentiate the strands on imaging modalities such as CT or MRI. For example, the multi-faceted marker 308 can correspond to the left side of the defect (e.g., defect 114), while the cylindrical marker 410 can correspond to the right side of the defect (e.g., defect 114), allowing a clinician to confirm proper positioning or detect displacement during postoperative imaging.

FIG. 5 depicts a perspective view of an example coupling member 506 linking a first strand 502 and a second strand 504 of the marker suture. The coupling member 506, first strand 502, and second strand 504 can be analogous to the any of the example coupling members, or strands described in connection with FIGS. 1-4. The example coupling member 506 is depicted as coupled to a first strand 502 via a linking member 524. The example coupling member 506 is depicted as coupled to a second strand 504 via a linking member 526.

In some embodiments, linking members 524 and 526 can each include a break or gap that allows the member to be opened and then be bent or pressed closed during surgery. In one implementation, linking member 524 can be preattached to the coupling member 506 prior to placement. In such implementations, linking member 526 remains open and is bent or pressed closed during surgery to secure the first strand 202 and the second strand 204. In another implementation, linking member 526 can be preattached to the coupling member 506 prior to placement, with linking member 524 remaining open and subsequently bent or pressed closed to complete the assembly. In yet another variation, both linking members 524 and 526 can be closed around, and the coupling member 506 can be open to allow attachment to linking member 524 and 526. After the attachment, the coupling member 506 can be bent or pressed closed to secure the strands 502 and 504 in place. This flexibility in configuration can facilitate quick and reliable intraoperative placement while decreasing manipulation of the surrounding tissue and eliminating the need for additional fastening devices. It also allows the marker suture assembly to accommodate movements (e.g., peristalsis, respiration, and weight loss) without placing tension on the tissue (e.g., mesenteric) through which the strands are woven.

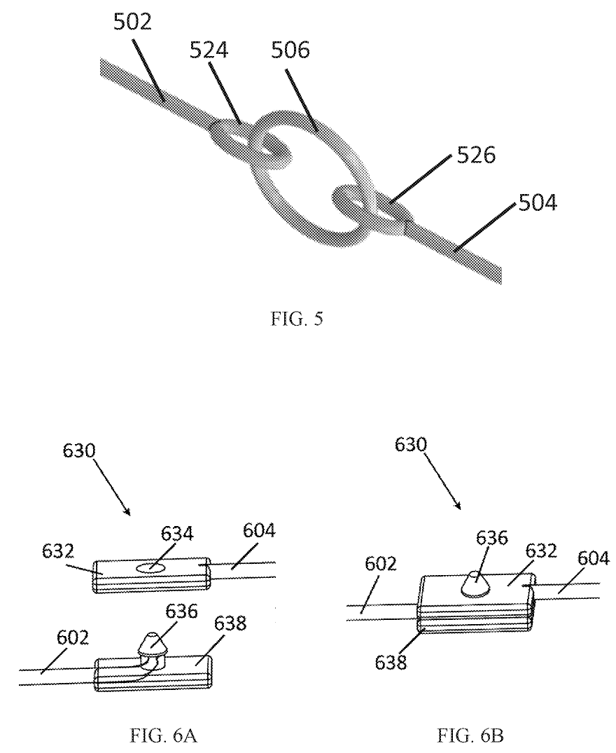

FIG. 6A depicts a perspective view of another example of a coupling member 630 in a disconnected configuration. FIG. 6A depicts a coupling member having a first side 638 coupled to a first strand and a second side 632 coupled to a second strand. The first side 638 includes a post 636 having a ridge. The second side 632 include a hole therethrough sized to be slight smaller in diameter than the ridge of the post 636. In some embodiments, this configuration can allow the post 636 to be pressed into the hole 634 with an interference fit during assembly. In some embodiments, the dimensions of the post 636, the ridge, and the hole 634 can be selected to permit a secure connection while still allowing relative rotational movement between the first side 638 and the second side 632 once coupled. For example, the first strand 602 and second strand 604 can remain freely movable relative to one another in a swiveling or pivoting motion after the coupling member 630 is engaged.

FIG. 6B depicts a perspective view of the example coupling member 630 of FIG. 6A in a connected configuration linking a first strand 602 and a second strand 604. In this connected state, the post 636 is seated within the hole of the second side 632, with the ridge engaging the internal diameter to hold the coupling closed. However, because the connection permits a controlled swivel or pivot motion, the first strand 602 and second strand 604 can rotate or shift slightly relative to each other. This flexibility allows the marker suture to accommodate bodily movements (e.g., peristalsis, respiration, or postoperative tissue movement) without applying tension to the tissue through which the strands are woven.

### Methods

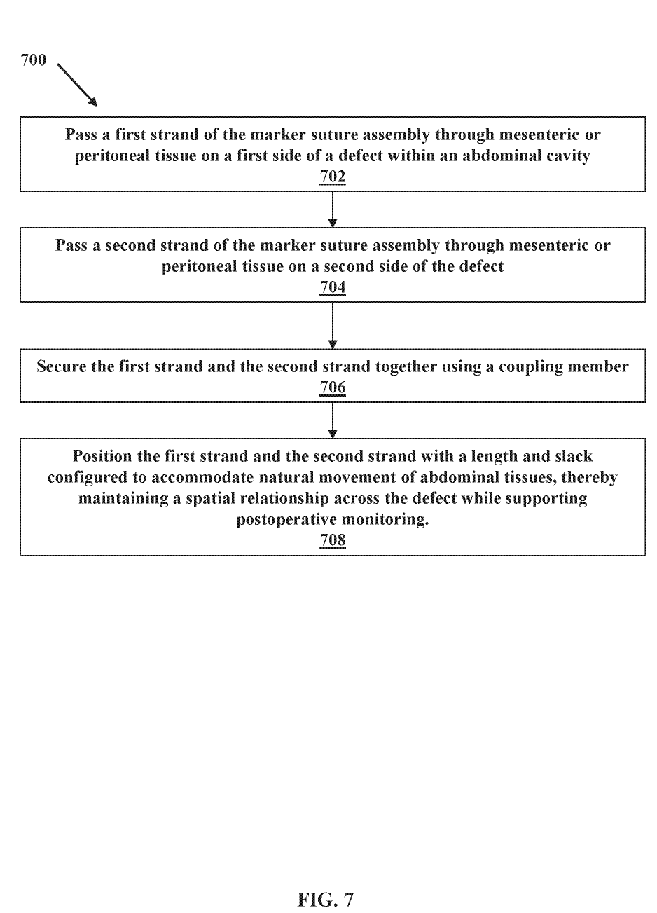

The methods described herein are carried out during intra-abdominal surgery. In some embodiments, the methods are performed in connection with procedures involving the mobilization, resection, or rearrangement of bowel or other abdominal structures, where the creation or closure of a defect within the abdominal cavity can occur. FIG. 7 shows a flow chart of an example method 700 for positioning a marker suture assembly during intra-abdominal surgery.

In some embodiments, a method of positioning a marker suture can include (702) passing a first strand (e.g., first strand 102, 202, 502, 602) through tissue (e.g., mesenteric or peritoneal tissue) on a first side of a defect (e.g., defect 114) within an abdominal cavity and (704) passing a second strand (e.g., second strand 104, 204, 504, 604) through tissue (e.g., mesenteric or peritoneal tissue) on a second side of the defect.

The method can further include (706) securing the first strand and the second strand together using a coupling member (e.g., coupling member 506, 630) to maintain the strands in a joined configuration. The method can further include (708) positioning the first strand and the second strand with a length and slack configured to accommodate natural movement of abdominal tissues, such as peristalsis, respiration, or tissue remodeling, while maintaining a spatial reference across the defect. For example, in some implementations, the first strand can include integrated radiopaque markers having a first geometric shape (e.g., multifaceted radiopaque marker 308 of FIG. 3) and the second strand can include integrated radiopaque markers having a second, different geometric shape (e.g., cylindrical radiopaque marker 410 of FIG. 4), such that the positioning of the strands can be confirmed postoperatively using imaging modalities such as CT or MRI.

In some implementations, the intra-abdominal surgery can be performed using an open, laparoscopic, endoscopic, or robotic-assisted approach. In further implementations, the defect can comprise an anchor suture made of dissolvable surgical thread. In some embodiments, the coupling member used to join the strands can be configured to be bent or pressed closed during surgery (e.g., coupling member 206, 506), allowing the marker suture assembly to flexibly accommodate physiological movements after placement.

In some embodiments, any of the marker sutures disclosed herein can be surgically positioned during or after a gastric bypass procedure. For example, in some implementations a marker suture having a first strand with a first geometric shape of integrated radiopaque markers and a second strand with a second geometric shape of integrated radiopaque markers can be surgically positioned with one strand on either side of a defect created between the Roux limb and transverse mesocolon during a gastric bypass procedure.

In some embodiments, any of the marker sutures disclosed herein can be surgically positioned during or after a gastric bypass procedure. For example, in some implementations, a marker suture having a first strand with a first geometric shape of integrated radiopaque markers and a second strand with a second geometric shape of integrated radiopaque markers can be surgically positioned with one strand on either side of a defect created between the created at the jejunojejunostomy anastomosis.

In another embodiment, any of the marker sutures disclosed herein can be surgically positioned during or after a small bowel resection procedure. For example, in some implementations, a marker suture having a first strand with a first geometric shape of integrated radiopaque markers and a second strand with a second geometric shape of integrated radiopaque markers can be positioned with one strand on either side of a defect created during resection and anastomosis of the small intestine.

In some embodiments, any of the marker sutures disclosed herein can be surgically positioned during or after a colorectal procedure. For example, in some implementations, a marker suture having a first strand with a first geometric shape of integrated radiopaque markers and a second strand with a second geometric shape of integrated radiopaque markers can be positioned with one strand on either side of a defect created during mobilization and resection of a segment of colon.

In some embodiments, any of the marker sutures disclosed herein can be surgically positioned during or after a hernia repair procedure. For example, in some implementations a marker suture having a first strand with a first geometric shape of integrated radiopaque markers and a second strand with a second geometric shape of integrated radiopaque markers can be positioned with one strand on either side of a defect created to reinforce the closure of fascial planes during repair of a hernia (e.g., parastomal or ventral hernia).

In some embodiments, any of the marker sutures disclosed herein can be surgically positioned during or after an oncologic resection procedure. For example, in some implementations a marker suture having a first strand with a first geometric shape of integrated radiopaque markers and a second strand with a second geometric shape of integrated radiopaque markers can be positioned with one strand on either side of a defect formed during tumor debulking procedures or segmental bowel resections in cytoreductive surgery.

In some embodiments, any of the marker sutures disclosed herein can be surgically positioned during or after a procedure to repair abdominal trauma. For example, in some implementations, a marker suture having a first strand with a first geometric shape of integrated radiopaque markers and a second strand with a second geometric shape of integrated radiopaque markers can be positioned with one strand on either side of a defect created during emergency bowel repair following abdominal trauma.

In some embodiments, any of the marker sutures disclosed herein can be surgically positioned during or after a gastrointestinal surgery (e.g., duodenal switch, gastrectomy). For example, in some implementations, a marker suture having a first strand with a first geometric shape of integrated radiopaque markers and a second strand with a second geometric shape of integrated radiopaque markers can be positioned with one strand on either side of a defect formed during procedures such as duodenal switch or subtotal gastrectomy.

### Other Embodiments

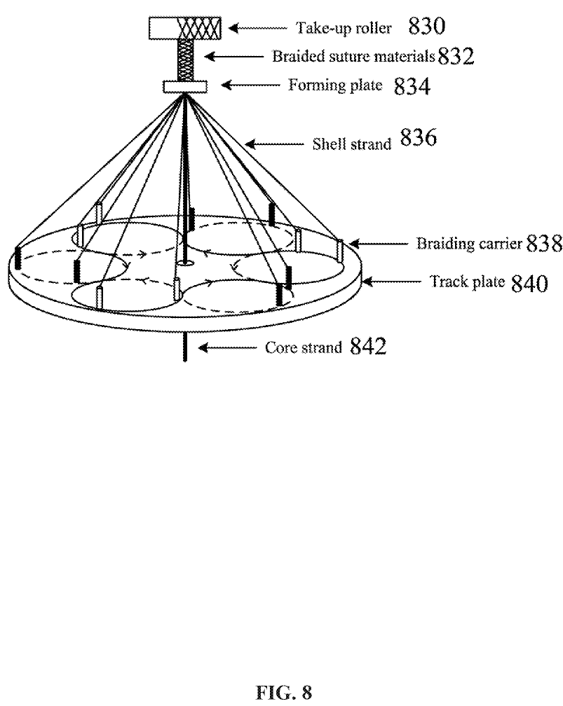

As described herein, the strands (e.g., first strand 102, 202, 502, 602, second strand 104, 204, 504, 604) can be made with surgical thread (e.g., any biocompatible non-dissolvable surgical thread). In some embodiments, the strands (e.g., any of the strands described herein) can be formed as a braided multi-strand suture having integrated radiopaque markers. FIG. 8 illustrates an example system configured to form such a multi-strand braided suture. The system includes a track plate 840 supporting a plurality of braiding carriers 838. The braiding carriers 838 are configured to weave strands 836 (e.g., first strand 102, 202, 502, 602, second strand 104, 204, 504, 604) around a central axis defined by a core strand 842. The shell strands 836 and the core strand 842 are drawn upward through a forming plate 834 to create the resulting braided suture materials 832, which are subsequently collected by a take-up roller 830. In some implementations, more than one braided multi-strand suture having integrated radiopaque markers can be used. For example, a first core strand (e.g., 832) can be formed with a first shape of radiopaque marker (e.g., 108, 110, 208, 210, 308, 410, 900, 1000, 1100) and a second core strand (e.g., 832) can be formed with a second shape of radiopaque marker. In such implementations, the first shape and the second shape can be different geometric shapes such that the first strand and the second strand can be differentiated under imaging.

During this manufacturing process, radiopaque markers, such as beads or coils, are added to the core strand 842 in predetermined increments. The shell strands 836 are then braided over the core strand 842 to creating a braid that internally encases the radiopaque elements. Because the radiopaque elements are integrated within a central lumen of the braid rather than applied as external protrusions, the outer surface of the braided suture materials 832 remains substantially smooth. This internal integration provides the advantage of minimizing tissue drag and trauma as the suture is passed through tissue, while simultaneously supporting knot security. In further embodiments, metallic wire strands may be woven in and out of the braided fibers. In some implementations, a polymeric outer coating may be applied over the braided structure and enclosed radiopaque elements to ensure the exterior profile remains smooth while maintaining overall flexibility and radiopacity.

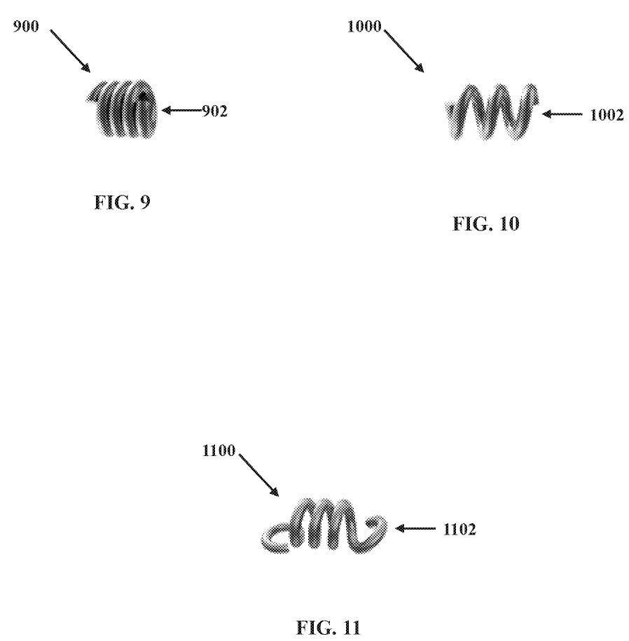

As described herein, the radiopaque markers 108, 110, 208, 210, 308, 410, can be formed from a variety of radiopaque materials and configured to have distinct geometries, such as cylindrical or multifaceted shapes, to improve visibility and orientation on medical imaging, other shapes are possible. In some embodiments, any of the radiopaque markers described herein can be included into any of the strand embodiments described herein. In some embodiments, the geometry of the radiopaque markers can be a ring, a coil, a spring, a spiral, and/or a spherical bead. For example, FIG. 9 depicts a perspective view of an example radiopaque marker 900 configured as a coil. The radiopaque marker 800 includes a central lumen 902 extending axially therethrough. This central lumen 902 is configured to accommodate a strand, such as the core strand 842, which passes through the central lumen 902 during formation. In some embodiments, the coil 900 may comprise a plurality of turns, or it may comprise fewer coils, such as a single ring. FIG. 10 depicts a perspective view of another example radiopaque marker 1000 configured as a spring. The radiopaque marker 1000 includes a central lumen 1002 extending axially therethrough, where the core strand 842 is similarly routed. FIG. 11 depicts a perspective view of another example radiopaque marker 1100 configured as a spiral. The radiopaque marker 1100 likewise includes a central lumen 1102 configured to receive the core strand 842.

For example, any of the radiopaque markers described herein and radiopaque markers 800, 900, or 1000 can be intermittently positioned along the core strand 842 such that the core strand 842 extends through their respective central lumens 802, 902, or 1002. The markers are then internally encased by the shell strands 836, providing predictable and visible increments of radiopacity along the length of the marker suture while allowing the outer surface of the suture to remain substantially smooth and minimize tissue drag.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A marker suture assembly comprising:
a first strand comprising a plurality of integrated radiopaque markers having a first geometric shape;
a second strand comprising a plurality of integrated radiopaque markers having a second geometric shape, wherein the second geometric shape is different from the first geometric shape; and
a coupling member configured to join the first strand and the second strand such that the first strand and the second strand are positioned on opposite sides of a defect within an abdominal cavity of a subject, wherein the first geometric shape and the second geometric shape are configured to allow visual differentiation between the first strand and the second strand under medical imaging.

2. A marker suture assembly comprising:
a first strand comprising a plurality of integrated radiopaque markers having a first geometric shape configured for positioning on a first side of a defect within an abdominal cavity of a subject;
a second strand comprising a plurality of integrated radiopaque markers having a second geometric shape configured for positioning on a second side of the defect within the abdominal cavity of the subject, wherein the second geometric shape is different from the first geometric shape; and
a coupling member configured to join the first strand and the second strand,wherein the coupling member is further configured to maintain the first strand and the second strand in a joined configuration while permitting rotational movement of the first strand relative to the second strand after coupling.

3. The marker suture assembly of claim 1, wherein the first geometric shape is a multi-faceted shape.

4. The marker suture assembly of claim 1, wherein the second geometric shape is a cylindrical shape.

5. The marker suture assembly of claim 1, wherein the integrated radiopaque markers of the first strand and the integrated radiopaque markers of the second strand are made from one or more materials selected from the group consisting of barium sulfate, tantalum oxide, zirconium dioxide, bismuth subcarbonate, bismuth trioxide, iodinated polymers, hydroxyapatite, calcium-based compounds, and titanium dioxide.

6. The marker suture assembly of claim 1, wherein the integrated radiopaque markers of the first strand and the integrated radiopaque markers of the second strand are made from titanium dioxide.

7. The marker suture assembly of claim 1, wherein the defect is one or more of a mesenteric defect, a peritoneal defect, a surgically created opening following bowel resection, a surgical anastomosis site, or a fascial defect created during hernia repair.

8. The marker suture assembly of claim 1, wherein the subject is a human or animal and the marker suture assembly is placed during intra-abdominal surgery.

9. The marker suture assembly of claim 1, wherein the coupling member comprises a flexible body configured to be bent or pressed closed to join the first strand and the second strand.

10. The marker suture assembly of claim 1, wherein the coupling member is formed in a ring shape configured to encircle and secure portions of the first strand and the second strand.

11. The marker suture assembly of claim 1, wherein the first strand and the second strand each further comprise a linking member configured to attach to the coupling member.

12. The marker suture assembly of claim 11, wherein each of the linking members are configured to allow rotational movement of the first strand relative to the coupling member after attachment.

13. The marker suture assembly of claim 12, wherein the coupling member and the respective linking members are collectively configured to maintain an attachment of the first strand and the second strand while permitting relative rotational movement between the first strand, the second strand, and the coupling member.

14. The marker suture assembly of claim 1, wherein the coupling member comprises a post extending from a first side and a hole formed through a second side, the hole being sized to engage the post with an interference fit to join the first strand and the second strand.

15. A method of positioning a marker suture assembly during intra-abdominal surgery, the method comprising:
passing a first strand of the marker suture assembly through mesenteric or peritoneal tissue on a first side of a defect within an abdominal cavity;
passing a second strand of the marker suture assembly through mesenteric or peritoneal tissue on a second side of the defect;
securing the first strand and the second strand together using a coupling member; andpositioning the first strand and the second strand with a length and slack configured to accommodate natural movement of abdominal tissues, thereby maintaining a spatial relationship across the defect while supporting postoperative monitoring.

16. The method of claim 15, wherein the first strand and the second strand are positioned with sufficient slack to accommodate postoperative changes in the abdominal cavity, including peristalsis, respiration, weight loss, or tissue remodeling.

17. The method of claim 15, further comprising imaging the first strand and the second strand after placement, wherein the imaging detects differently shaped integrated radiopaque markers on the first strand and the second strand to assess the spatial positioning across the defect.

18. The method of claim 15, wherein the intra-abdominal surgery is performed using one or more modalities selected from open surgery, laparoscopic surgery, endoscopic surgery, or robotic-assisted surgery.

19. The method of claim 15, wherein the defect comprises an anchor suture formed from dissolvable surgical thread used to close the defect.

20. The method of claim 15, wherein the coupling member is configured to be bent or pressed closed during surgery to join the first strand and the second strand while permitting relative movement between the first strand and the second strand after placement.
